(19) United States
(12) Patent Application Publication
(10) Pub. No.: US 2026/0083931 A1
(43) Pub. Date: Mar. 26, 2026

(54) RELAXATION SYSTEM

(71) Applicant: Calm and Sense Technologies, LLC, Palm Beach Gardens, FL (US)

(72) Inventors: Daniel Shuter, New York, NY (US); Angela Sun, New York, NY (US); David Shuter, Palm Beach Gardens, FL (US)

(21) Appl. No.: 19/206,196

(22) Filed: May 13, 2025

Related U.S. Application Data
(62) Division of application No. 18/734,004, filed on Jun. 5, 2024, now abandoned.

(51) Int. Cl. A61M 21/00

(52) U.S. Cl. CPC A61M 21/00; A61M 2021/0016; A61M 2021/0022; A61M 2021/0066

(57) ABSTRACT

A relaxation system includes an enclosure, a vibrator, electrodes, a user feedback stimulus generator, and at least one processing device in electrical communication with the vibrator, the electrodes and the user feedback stimulus generator. The vibrator is configured to induce vibrations of the enclosure. Each electrode includes a contact surface flush with or extending outwardly from an outer surface of the enclosure. The user feedback stimulus generator is configured to generate a user-perceptible stimulus different than that of the vibrator. The processing device is configured to control the vibrator based on signals received from the electrodes, to measure at least one cardiac metric based on signals received from the electrodes and provide a detected cardiac metric, to determine a relaxation status by comparing the detected cardiac metric to a target cardiac metric, and to control operation of the user feedback stimulus generator based on the relaxation status.

### BACKGROUND

Heart rate variability (HRV) represents fluctuations in the time intervals between successive heartbeats. A higher HRV is known to be associated with an optimal state for inducement of relaxation and sleep. Appropriate slow-paced breathing has been found to increase one's HRV. A person's resonance frequency is the respiration cycle that produces the greatest HRV.

Devices exist to measure HRV and literature describes sleep aid and relaxation devices that include sensors to detect a user's HRV along with the user's breath pattern. For example, US 2024/0001069 A1 discloses a sleep aid apparatus that includes sensors for detecting a user's HRV and US 2020/0245931 A1 discloses stretch receptors on a vest, which is worn by the user, to measure ventilation or respiration rate. The use of stretch receptors to measure ventilation or respiration rate does not lend itself to a relatively small device for guiding a user's respiration cycle toward her resonance frequency. Moreover, determining a respiration phase through the use of a sleep aid apparatus may very well unnecessarily complicate the apparatus when the goal to achieving the optimal state for inducement of relaxation and sleep is to increase the user's HRV toward the maximum.

### SUMMARY

In view of the foregoing, a relaxation system includes and enclosure, a vibrator disposed in the enclosure, electrodes mounted to the enclosure, a user feedback stimulus generator mounted to the enclosure, and at least one processing device in electrical communication with the vibrator, the electrodes and the user feedback stimulus generator. The vibrator is configured to induce vibrations of the enclosure. Each electrode includes a contact surface flush with or extending outwardly from an outer surface of the enclosure. The user feedback stimulus generator is configured to generate a user-perceptible stimulus different than that of the vibrator. The processing device is configured to control the vibrator, to measure at least one cardiac metric based on signals received from the electrodes and provide a detected cardiac metric, to determine a relaxation status by comparing the detected cardiac metric to a target cardiac metric stored in a database, and to control operation of the user feedback stimulus generator based on the relaxation status.

### BRIEF DESCRIPTION OF THE DRAWINGS

FIG. 1 is a schematic depiction of a relaxation system.

FIG. 2 is a schematic depiction of an enclosure of the relaxation system resting on a user's sternum.

FIG. 3 is a perspective view of an example of the enclosure.

FIG. 4 is a flow diagram depicting an example of a method of operating the relaxation system.

### DETAILED DESCRIPTION

The detailed description and specific examples, while describing particular embodiments, are intended for purposes of illustration only and are not intended to limit the scope of the invention. These embodiments and other features, aspects, and advantages will become better understood from the following description, appended claims, and accompanying drawings. The figures are merely schematic and are not drawn to scale, and the same reference numerals are used throughout the figures to indicate the same or similar parts.

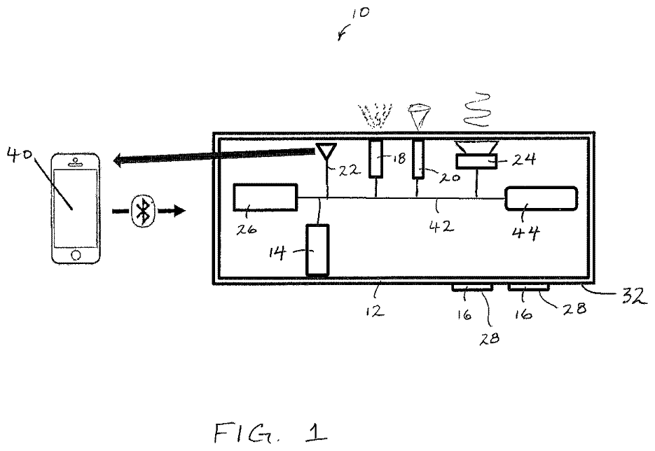

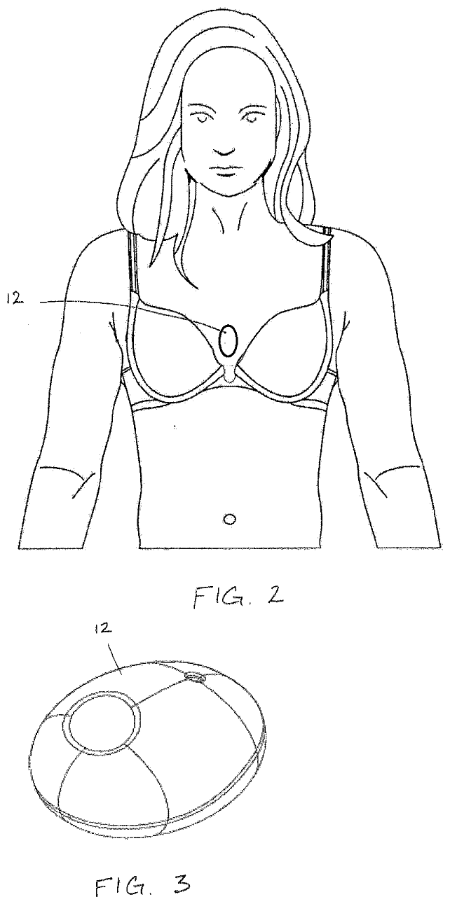

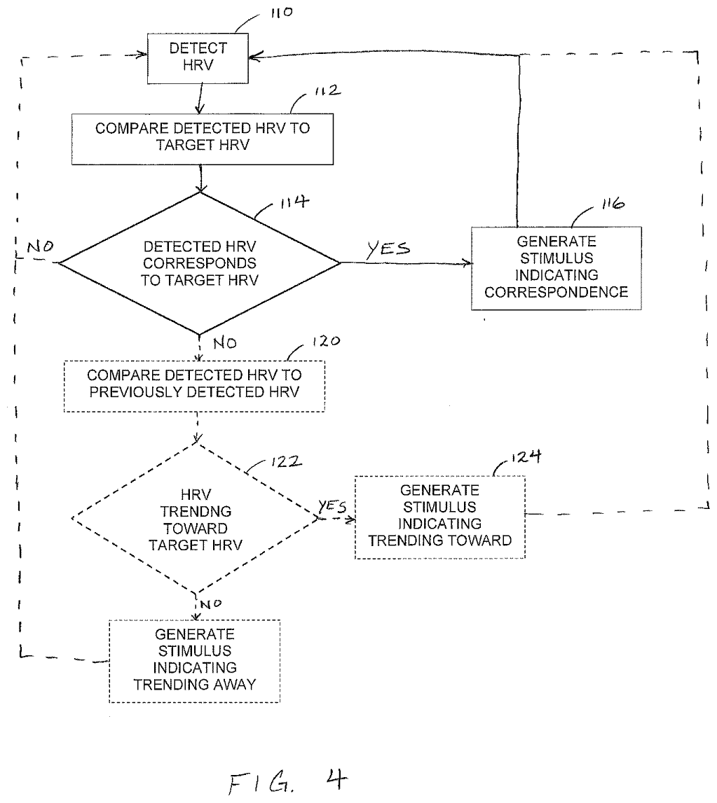

FIG. 1 depicts a relaxation system 10 including an enclosure 12 that is intended to rest on a person's sternum (see FIG. 2) for guiding a user in paced breathing to encourage relaxation and/or sleep induction. The enclosure 12 can take the form shown in FIG. 3; however, the enclosure 12 can take other forms as well. Accordingly, the enclosure 12 is schematically depicted in FIG. 1.

With reference to FIG. 1, the relaxation system 10 includes a vibrator 14 disposed in the enclosure 12, electrodes 16 mounted to the enclosure 12, at least one user feedback stimulus generator 18, 20, 22, 24 mounted to the enclosure, and at least one processing device, e.g., a data processing unit 26, in electrical communication with the vibrator 14, the electrodes 16 and the at least one user feedback stimulus generator 18, 20, 22, 24. The vibrator 14 is configured to induce vibrations of at least a portion of the enclosure 12, which can provide a user-perceptible stimulus to the user's sternum. Each electrode 16 includes a respective contact surface 28 flush with or extending outwardly from an outer surface 32 of the enclosure 12. The user feedback stimulus generators 18, 20, 22, 24 are configured to generate a user-perceptible stimulus different than that of the vibrator 14. The at least one processing device, which can include the data processing unit 26, is configured to control the vibrator 14 based on signals received from the electrodes 16, to measure at least one cardiac metric based on signals received from the electrodes 16 and provide a detected cardiac metric, to determine a relaxation status by comparing the detected cardiac metric to a target cardiac metric stored in a database associated with the at least one processing device, and to control operation of the at least one user feedback stimulus generator 18, 20, 22, 24 based on the relaxation status.

The vibrator 14 is disposed in the enclosure 12 and is mounted in the enclosure 12 in a manner such that the vibrator 14 induces vibrations of at least a portion of the enclosure 12, which can provide a user-perceptible stimulus to the user's sternum. For example, the enclosure may include a more resilient section that can vibrate with respect to a more rigid portion of the enclosure, or the entire enclosure may vibrate. Examples of the vibrator 14 include a wideband LRA (linear resonant actuator) motor, an ERM (eccentric rotating mass) motor, and speaker voice coils. It may be desirable to operate the vibrator 14 at a frequency below 60 Hz so that the operation of the vibrator 14 is not perceived audibly by the user.

The electrodes 16 can be dry ECG (electrocardiogram) biopotential electrodes that do not require the typical electrolytic conductive gel and skin preparation as compared to wet electrodes. At least two, and perhaps more, electrodes 16 can be mounted to the enclosure 12 and in electrical communication with the at least one processing unit 26. The electrodes 16 are mounted to the enclosure 12 in a manner such that when the enclosure 12 is placed on the user's sternum (see FIG. 2) ionic currents from the user's body surface are converted into electrical signals for later processing by the at least one processing device.

The user feedback stimulus generators 18, 20, 22, 24 are mounted to the enclosure 12 and are configured to generate a user-perceptible stimulus different than that of the vibrator 14. As illustrated in FIG. 3, the user feedback stimulus generators 18, 20, 22, 24 are mounted within the enclosure 12; however, one or both user feedback stimulus generators 18, 20, 22, 24 may be mounted on an exterior of the enclosure 12, e.g., mounted to the outer surface 32 of the enclosure 12. Providing the user feedback stimulus generators 18, 20, 22, 24 mounted to the enclosure 12 provides a compact device that can guide a user in paced breathing to encourage relaxation and/or sleep induction. That the user feedback stimulus generators 18, 20, 22, 24 provide a user-perceptible stimulus different than that of the vibrator 14 allows for multiple sensory cues to help users practice breathwork effectively.

The user feedback stimulus generators 18, 20, 22, 24 can include a first user feedback stimulus generator 18, which can be in the form of a scent delivery mechanism, a second user feedback stimulus generator 20, which can be in the form of a radiation delivery mechanism, a third user feedback stimulus generator 22, which can be in the form of a wireless signal generator which can be in electrical communication with a mobile computing device such as a smartphone 40, and a fourth user feedback stimulus generator 24, which can be in the form of a speaker. The scent delivery mechanism can be a fluid-based diffuser that atomizes a fluid to eject an atomized fluid, which can be smelled by the user, from the scent delivery mechanism. The radiation delivery mechanism can be configured to generate radiation in the form of heat or visible light. The signal generator can provide signals to the smartphone 40, e.g., via a wireless connection, which can be processed by a processor on and/or a software application running on the smartphone 40 to display content to the user. The speaker can generate sounds via communication with the data processing unit 26.

FIG. 1 depicts the data processing unit 26 positioned within the enclosure 12. The data processing unit 26 positioned within the enclosure 12 can be a component within a data processing system. For example, the data processing unit 26 positioned within the enclosure 12 can be in electrical communication with a mobile computing device such as the smartphone 40. The data processing unit 26 can be provided on a main circuit board (not shown in FIG. 3) provided within the enclosure 12. If desired, additional circuit boards, e.g. a daughter circuit board (also not shown) may be in electrical communication with the electrodes 16 and the main circuit board to provide filtering and signal conditioning to signals generated by the electrodes 16. A bus 42 or similar electrical connection may be provided to electrically connect the vibrator 14, the electrodes 16 and the user feedback stimulus generators 18, 20, 22, 24 with each other and to a power source 44, which can be a rechargeable battery. The data processing unit 26 and other data processing devices that make up the at least one processing device of the data processing system can be implemented with software and/or hardware, to perform the various functions described herein. Examples of circuitry that may be employed include conventional microprocessors, application specific integrated circuits (ASICs), and field-programmable gate arrays (FPGAs). Also the data processing system may be associated with one or more storage media such as volatile and non-volatile computer memory such as RAM, PROM, EPROM, and EEPROM.

The at least one processing device, which can include the data processing unit 26 and/or the processor found in the smartphone 40, is configured to control the vibrator 14. As an example, the vibrator 14 can be controlled to match the inhalation phase and exhalation phase of a desired breathing pattern where the vibrator 14 is turned on during the inhalation phase and exhalation phase of the desired breathing pattern, and the vibrator 14 is turned off during pauses between the inhalation phase and exhalation phase of the desired breathing pattern.

The at least one processing device, which can include the data processing unit 26 and/or the processor found in the smartphone 40, can be further configured to control the vibrator 14 based on signals received from the electrodes 16. For example, the duration of time that the vibrator 14 is turned on or off can be adjusted based on signals received from the electrodes 16 to provide indications the user of when to inhale and to exhale to guide the user's respiration cycle toward the user's resonance frequency.

The at least one processing device, which can include the data processing unit 26 and/or the processor found in the smartphone 40, can be further configured to measure at least one cardiac metric based signals received from the electrodes 16. Based on the signals received from the electrodes 16, the at least one processing device can output a detected cardiac metric. Among the cardiac metrics that can be measured based signals received from the electrodes 16 are the following: heart rate (HR), heart rate variability (HRV), respiratory sinus arrhythmia (RSA), power and frequency. Heart rate is the number of heartbeats per minute. HRV is the fluctuation in the time intervals between adjacent heartbeats. HRV can be described using time domains and frequency domains. RSA refers to the respiration-driven speeding and slowing of the heart via the vagus nerve based signals. Power is the signal energy found within a frequency band, which can be expressed in absolute or relative power. Frequency measurements can estimate the distribution of absolute or relative power into frequency bands, e.g., ultra-low-frequency (≤0.003 HZ), very-low-frequency (0.0033-0.04 Hz), low-frequency (0.04-0.15 Hz), and high-frequency (0.15-0.4 Hz).

The at least one processing device, which can include the data processing unit 26 and/or the processor found in the smartphone 40, can be further configured to determine a relaxation status by comparing the detected cardiac metric to a target cardiac metric stored in a database associated with the at least one processing device. In a particular example where the measured cardiac metric is HRV, by comparing the detected HRV to the target HRV, the at least one processing device can determine whether the user's detected HRV is trending towards the target HRV, is trending away from the target HRV or is corresponding with the target HRV. Thus, the user's HRV status can be determined to be one of trending towards, trending away or in correspondence with the target HRV. In a similar fashion, by comparing the detected cardiac metric (e.g., HR, RSA, power or frequency) to the target cardiac metric, the at least one processing device can determine whether the user's detected cardiac metric is trending towards the target cardiac metric, is trending away from the target cardiac metric or is corresponding with the target cardiac metric.

The at least one processing device, which can include the data processing unit 26 and/or the processor found in the smartphone 40, can be further configured to control operation of the user feedback stimulus generators 18, 20, 22, 24 based on the relaxation status. The user feedback stimulus generators 18, 20, 22, 24 can generate a user-perceptible stimulus different than that of the vibrator 14, so that the user can easily perceive one stimulus from another, to guide the user toward a respiration cycle that produces the greatest HRV, for example. The user feedback stimulus generators 18, 20, 22, 24 can generate a user-perceptible stimulus different than that of the vibrator 14, so that the user can easily perceive one stimulus from another, to guide the user toward a respiration cycle that produces the desired cardiac metric, which can be stored in the database.

For example, the at least one processing device, which can include the data processing unit 26 and/or the processor found in the smartphone 40, can be further configured to control operation of the user feedback stimulus generators 18, 20, 22, 24 to vary the user-perceptible stimulus based on the relaxation status. As a further example, the at least one processing device can be configured to control operation of the user feedback stimulus generators 18, 20, 22, 24 to signal the user feedback stimulus generators 18, 20, 22, 24 to generate a first user-perceptible stimulus when the detected HRV is trending towards the target HRV or, more generally, when the detected cardiac metric is trending towards the target cardiac metric. In a particular example where HRV is measured, the at least one processing device can be configured to control operation of the user feedback stimulus generators 18, 20, 22, 24 to signal the user feedback stimulus generators 18, 20, 22, 24 to generate the first user-perceptible stimulus, which may be lesser in intensity as compared to a second user-perceptible stimulus, which can be generated by the user feedback stimulus generators 18, 20, 22, 24 when the HRV status is found to be corresponding with the target HRV or even further trending towards the target HRV. For example, the volume of atomized fluid ejected from the first user feedback stimulus generator 18 (e.g., scent delivery mechanism) can increase as the detected HRV trends closer to the target HRV. Similarly, the intensity of radiation, whether in the form of heat or visible light, emitted from the second user feedback stimulus generator 20 (e.g., radiation delivery mechanism) can increase or decrease as the detected HRV trends closer to the target HRV. Also, in the case where visible light is emitted from the radiation delivery mechanism, the color of visible light emitted from the radiation delivery mechanism can change as the detected HRV trends closer to or further away from the target HRV.

In an even further example, the at least one processing device can be configured to control operation of the user feedback stimulus generators 18, 20, 22, 24 to signal the user feedback stimulus generators 18, 20, 22, 24 to change or to increase an intensity of the user-perceptible stimulus being generated when the detected cardiac metric corresponds with the target cardiac metric. As mentioned above, the at least one processing device can control operation of the user feedback stimulus generators 18, 20, 22, 24 to signal the user feedback stimulus generators 18, 20, 22, 24 to generate a first user-perceptible stimulus when the detected HRV is trending towards the target HRV. When the detected HRV corresponds with the target HRV, the at least one processing device can control operation of the user feedback stimulus generators 18, 20, 22, 24 to signal the user feedback stimulus generators 18, 20, 22, 24 to change or to increase the intensity of the user-perceptible stimulus from the first user-perceptible stimulus to a different, e.g., more or less intense, second user-perceptible stimulus. In the specific instance that the at least one processing device is controlling operation of the radiation delivery mechanism, the color of visible light emitted from the radiation delivery mechanism can change as the detected HRV corresponds with the target HRV. In the instances where the detected HRV is found to correspond with the target HRV, this correspondence does not need to be an exact match between the detected HRV and the target HRV; instead, the correspondence could be within an acceptable range.

The at least one processing device, which can include the data processing unit 26 and/or the processor found in the smartphone 40, can be further configured to receive user input instructions for controlling when the user feedback stimulus generators 18, 20, 22, 24 are to deliver the user-perceptible stimulus. For example, the at least one processing device can be configured to receive user input instructions for controlling whether scent or radiation is being delivered as the user-perceptible stimulus. As mentioned above, a software application running on the smartphone 40 can be in wireless communication with the data processing unit 26. The software application can be designed to receive user input to allow the user to decide the type of user-perceptible stimulus to be generated and the basis for the generation of the user-perceptible stimulus. For example, a user who prefers a scent to be delivered may, via the software application, instruct that a scent be delivered by the first user feedback stimulus generator 18 when the user's HRV (or relaxation) status is determined to be trending towards the target HRV and that a particular color light be generated by the second user feedback stimulus generator 20 when the user's HRV status is determined to be in correspondence with the target HRV. The preceding sentence is only one example of numerous other manners in which the user may customize the user-perceptible stimulus to be generated based on the user's relaxation status through the software program. By providing the user the ability to customize the user-perceptible stimulus to be generated, the user has the ability to customize the relaxation system in a manner that works best for the user to encourage the user toward the user's resonance frequency or to encourage the user toward the target cardiac metric, whether that cardiac metric be HR, HRV, RSA, power or frequency.

Another example is that the at least one processing device, which can include the data processing unit 26 and/or the processor found in the smartphone 40, can be further configured to control operation of the first user feedback stimulus generator 18, which is a scent delivery mechanism in this example, to control an intensity of scent being generated by the scent delivery mechanism based on based on the relaxation status. For example, the intensity of scent being generated by the scent delivery mechanism may be increased, for example by increasing the volume of fluid being atomized, as the user's HRV status is determined to be trending towards the target HRV the closer the user's HRV status is to the target HRV status. In addition, the intensity of scent being generated by the scent delivery mechanism may be decreased, for example by decreasing the volume of fluid being atomized, or delivery may cease as the user's HRV status is determined to be trending away from the target HRV. This particular control of the scent delivery may be pre-programmed in the at least one processing device or the software program, or the user may have the ability to customize the scent delivery through queries presented to the user on the smartphone 40 and the software program.

Another example is that the at least one processing device, which can include the data processing unit 26 and/or the processor found in the smartphone 40, can be further configured to control operation of the second user feedback stimulus generator 20, which is a radiation delivery mechanism in this example, to control an intensity of heat being generated by the radiation delivery mechanism based on based on the relaxation status. For example, the intensity of heat being generated by the radiation delivery mechanism may be increased, for example by increasing the power being deliver to the radiation delivery mechanism, as the user's HRV status is determined to be trending towards the target HRV the closer the user's HRV status is to the target HRV status. In addition, the intensity of heat being generated by the radiation delivery mechanism may be decreased, for example by decreasing the power being deliver to the radiation delivery mechanism, or delivery power may cease as the user's HRV status is determined to be trending away from the target HRV. In another example, the at least one processing device, which can include the data processing unit 26 and/or the processor found in the smartphone 40, can be further configured to control operation of the second user feedback stimulus generator 20, which is a radiation delivery mechanism in this example, to control operation of the radiation delivery mechanism to control an intensity or wavelength of light being generated by the radiation delivery mechanism based on based on the HRV status. For example, the intensity of light being generated by the radiation delivery mechanism may be increased as the user's HRV status is determined to be trending towards the target HRV the closer the user's HRV status is to the target HRV status. Alternatively, the wavelength of light being generated by the radiation delivery mechanism may be changed as the user's HRV status is determined to be trending towards the target HRV the closer the user's HRV status is to the target HRV status. In addition, the intensity of light being generated by the radiation delivery mechanism may be decreased or delivery of light may cease as the user's HRV status is determined to be trending away from the target HRV. Alternatively, the wavelength of light being generated by the radiation delivery mechanism may be changed or delivery of light may cease as the user's HRV status is determined to be trending away from the target HRV. The particular control of the radiation delivery described above may be pre-programmed in the at least one processing device or the software program, or the user may have the ability to customize the radiation delivery through queries presented to the user on the smartphone 40 and the software program.

The at least one processing device, which can include the data processing unit 26 and/or the processor found in the smartphone 40, can be further configured to operate in different modes. These different modes can include a sleep-inducing mode, a biofeedback training mode and a resonance frequency tracking mode. In general, when in the sleep-inducing mode the relaxation system 10 operates to control the vibrator 14 to correspond to a breathing pattern, or respiration cycle, to be matched by the user to encourage the user to breathe at the user's resonance frequency, which has been previously found or estimated through the program found on data processing unit 26 and/or the processor found in the smartphone 40. This breathing pattern, or respiration cycle should encourage synchronization between the detected HRV and the target HRV. In general, when in the resonance frequency tracking mode the relaxation system 10 operates to control the vibrator 14 to correspond to several different breathing patterns, or respiration cycles, to be matched by the user. When in the resonance frequency tracking mode, the user's breathing pattern is not tracked, however, the user's HRV is detected and tracked to locate the breathing pattern that produces the greatest HRV. In general, when in the biofeedback training mode the relaxation system 10 operates to control the vibrator 14 to correspond to a breathing pattern, or respiration cycle, to be matched by the user to encourage the user to breathe at the user's resonance frequency, which has been previously found in the same manner described above for the sleep-inducing mode. In addition, when in the biofeedback training mode the relaxation system 10 the at least one processing device controls the operation of the user feedback stimulus generators 18, 20, 22, 24 to vary the user-perceptible stimulus based on the user's HRV status in the manners already described above. By varying the user-perceptible stimulus based on the user's HRV status, the user-perceptible stimulus can be considered a “reward” being offered to the user as the user trends toward or corresponds with the breath pattern that produces the greatest HRV. This “reward” can be used as a training feature for the user to encourage synchronization between the detected HRV and the target HRV.

A method of operating the relaxation system 10 in the biofeedback training mode, for example, will be described with reference to FIG. 4. The process shown in FIG. 4 occurs while controlling the vibrator 14 disposed in the enclosure 12 to induce vibrations of the enclosure 12. The vibrator 14 can be controlled to match the inhalation phase and exhalation phase of a desired breathing pattern where the vibrator 14 is turned on during the inhalation phase and exhalation phase of the desired breathing pattern.

The process in FIG. 4 will be described with reference to measuring the user's HRV to determine if the detected HRV corresponds to a target HRV, but the process could be employed with measuring another cardiac metric such as HR, RSA, power or frequency. At 110, a person's HRV is detected via the electrodes 16 mounted to the enclosure 12 and the at least one processing device, which can include the data processing unit 26 and/or the processor found in the smartphone 40, that is in electrical communication with the electrodes 16. Typically, the enclosure 12 is placed on the user's sternum, as shown in FIG. 2, while the user is lying down on her back. As mentioned above, other cardiac metrics such as HR, RSA, power or frequency could also be detected at 110.

At 112, the detected HRV is compared to a target HRV via the at least one processing device, which can include software running on the data processing unit 26 and/or the processor found in the smartphone 40. The HRV target can be the person's greatest HRV, which is associated with the person's resonance frequency and is associated with the person's optimal state for relaxation and sleep. If desired, other detected cardiac metrics can be compared to the appropriate target cardiac measurement at 112.

At 114, if the detected HRV corresponds to the target HRV, then the process moves to 116 and an HRV correspondence user-perceptible stimulus, which is different than that of the vibrator, is generated via at least one of the user feedback stimulus generators 18, 20, 22, 24 mounted to the enclosure 12. The different types of HRV correspondence user-perceptible stimuli have been described above and can include scent, heat, light, sound and outputs displayed on the smartphone 40 (FIG. 1), for example. After the HRV correspondence user-perceptible stimulus is generated at 116, the process can return to 110 and the person's HRV can be detected via the electrodes 16 mounted to the enclosure 12 and the at least one processing device. Alternatively or additionally, if the detected cardiac metric corresponds to the target cardiac metric, then the process can move to 116 and a cardiac metric correspondence user-perceptible stimulus, which is different than that of the vibrator, can be generated.

If, at 114, the detected HRV does not correspond to the target HRV, then the process can return to 110 and detect the person's HRV via the electrodes 16 mounted to the enclosure 12 and the at least one processing device in electrical communication with the electrodes 16. In other words, no stimulus (other than the vibrations being generated by the vibrator) may be generated, which can indicate to the user that the target HRV has not been achieved. This return branch from 114 to 110 is shown in broken lines because the process may also follow another path if the detected HRV does not correspond to the target HRV. Also, if at 114, the detected cardiac metric does not correspond to the target cardiac metric, then the process can return to 110 and detect the person's cardiac metric via the electrodes 16.

If the detected HRV does not correspond to the target HRV, at 114, then, at 120, a later detected HRV is compared to a previously detected HRV. At 122, a determination is made whether the later detected HRV is trending towards the target HRV based on comparing the later detected HRV to the previously detected HRV. This determination can be made, for example, in the software running on the data processing unit 26 and/or the processor found in the smartphone 40. If the later detected HRV is closer to the target HRV as compared to the immediately previously detected HRV, then this can be an indication that the HRV is trending toward the target HRV. If the later detected HRV is further from the target HRV as compared to the immediately previously detected HRV, then this can be an indication that the HRV is trending away from the target HRV.

With continued reference to FIG. 4, the process further includes generating another user-perceptible stimulus different than that of the vibrator and different than that of the HRV correspondence user-perceptible stimulus prior to returning to detecting the person's HRV via the electrodes 16 mounted to the enclosure 12 and the at least one processing device in electrical communication with the electrodes 16. For example if the later detected HRV is determined to be trending towards the target HRV, at 122, then, at 124, the other user-perceptible stimulus is generated as a trending toward user-perceptible stimulus, which is different than that of the vibrator and different than that of the HRV correspondence user-perceptible stimulus. The process can then return to detecting the person's HRV via the electrodes 16 mounted to the enclosure 12 and the at least one processing device in electrical communication with the electrodes 16, at 110. If, however, at 122, the later detected HRV is determined to be trending away from the target HRV, then the other user-perceptible stimulus can be generated as a trending away user-perceptible stimulus, which is different than that of the vibrator, the HRV correspondence user-perceptible stimulus and the trending toward user-perceptible stimulus. As such, the user can receive different stimuli based on whether the user's HRV is trending towards or away from the target HRV. The process can then return to detecting the person's HRV via the electrodes 16 mounted to the enclosure 12 and the at least one processing device in electrical communication with the electrodes 16, at 110. These trends described above can also be employed with the other cardiac metrics that can be measured, such as HR, RSA, power or frequency.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. (canceled)

2. The relaxation system of claim 17, wherein the at least one processing device is further configured to control operation of the user feedback stimulus generator to vary the user-perceptible stimulus based on the relaxation status.

3. The relaxation system of claim 2, wherein the at least one processing device is further configured to control operation of the user feedback stimulus generator to signal the user feedback stimulus generator to deliver the user-perceptible stimulus when the detected cardiac metric is trending towards the target cardiac metric.

4. The relaxation system of claim 3, wherein the at least one processing device is further configured to control operation of the user feedback stimulus generator to signal the user feedback stimulus generator to change or to increase an intensity of the user-perceptible stimulus being delivered when the detected cardiac metric corresponds to the target cardiac metric.

5. (canceled)

6. The relaxation system of claim 17, wherein the user feedback stimulus generator includes at least one of a scent delivery mechanism, a radiation delivery mechanism, a wireless signal generator and a speaker.

7. The relaxation system of claim 6, wherein the at least one processing device is further configured to control operation of the scent delivery mechanism to control an intensity of scent being generated by the scent delivery mechanism based on based on the relaxation status.

8. The relaxation system of claim 6, wherein the at least one processing device is further configured to control operation of the radiation delivery mechanism to control an intensity of heat being generated by the radiation delivery mechanism based on based on the relaxation status.

9. The relaxation system of claim 6, wherein the at least one processing device is further configured to control operation of the radiation delivery mechanism to control an intensity or wavelength of light being generated by the radiation delivery mechanism based on based on the relaxation status.

10. The relaxation system of claim 6, wherein the at least one processing device is further configured to receive user input instructions for controlling whether scent, radiation or sound is being delivered as the user-perceptible stimulus.

11. The relaxation system of claim 17, wherein the at least one processing device is further configured to operate in at least one of a sleep-inducing mode and a biofeedback training mode,
when in the sleep-inducing mode, the at least one processing device controls the vibrator to correspond to a breathing pattern to be matched by the user,
when in the biofeedback training mode, the at least one processing device controls the vibrator to correspond to a breathing pattern to be matched by the user and the at least one processing device controls the operation of the user feedback stimulus generator to vary the user-perceptible stimulus based on the relaxation status.

12. The relaxation system of claim 17, wherein the detected cardiac metric is heart rate variability (HRV) and the target cardiac metric is a target HRV.

13-16. (canceled)

17. A relaxation system comprising:
an enclosure configured to rest on a person's sternum;
a vibrator disposed in the enclosure and configured to induce vibrations of at least a portion of the enclosure and provide a perceptible stimulus to the person's sternum;
electrodes mounted to the enclosure, each electrode including a contact surface flush with or extending outwardly from an outer surface of the enclosure, the electrodes being mounted to the enclosure in a manner such that when the enclosure is placed on the person's sternum ionic currents from the person's body surface are converted into electrical signals;
a user feedback stimulus generator mounted to the enclosure and configured to generate a user-perceptible stimulus that is different than that of the vibrator; and
at least one processing device in electrical communication with the vibrator, the electrodes and the user feedback stimulus generator, the at least one processing device being configured:
to control the vibrator;
to measure at least one cardiac metric based signals received from the electrodes and provide a detected cardiac metric;
to determine a relaxation status by comparing the detected cardiac metric to a target cardiac metric stored in a database associated with the at least one processing device; and
to control operation of the user feedback stimulus generator based on the relaxation status.

18. The relaxation system of claim 17, wherein the at least one processing device is further configured to receive user input instructions for controlling when the user feedback stimulus generator is to deliver the user-perceptible stimulus that is different than that of the vibrator.
